(19) United States
(12) Patent Application Publication
(10) Pub. No.: US 2026/0141998 A1
(43) Pub. Date: May 21, 2026

(54) METHOD FOR TRACKING, VERIFYING, AND AUTHENTICATING MULTI-STAGE CHAIN OF CUSTODY

(71) Applicant: Courtfact, LLC, Royal Oak, MI (US)

(72) Inventors: Jason Tizedes, Saline, MI (US); Tyson List, Gilbert, AZ (US); Daniel Kurin, Flushing, MI (US)

(73) Assignee: Courtfact, LLC, Royal Oak, MI (US)

(21) Appl. No.: 19/393,099

(22) Filed: Nov. 18, 2025

Related U.S. Application Data
(60) Provisional application No. 63/721,602, filed on Nov. 18, 2024.

(51) Int. Cl. G16H 10/40; A61B 5/00; A61B 5/11; A61B 90/96

(52) U.S. Cl. CPC G16H 10/40; A61B 5/1112; A61B 5/4845; A61B 5/6833; A61B 90/96

(57) ABSTRACT

A method for electronically tracking and verifying the application, removal, and chain of custody of wearable drug testing patches is provided. The method involves applying a drug testing patch to a wearer, electronically logging application events associated with the drug patch, including capturing electronic signatures from both the wearer and a first technician, and compiling an application chain of custody document for the respective drug patch that links client information, case record information, the patch serial number, and the respective electronic signature identifiers. After a predetermined wear duration, the drug patch is removed by a second technician, and a patch removal event is logged, including capturing electronic signatures from both the wearer and the second technician. A completed chain of custody document, including a barcode linked to the client, case record, and the patch is generated to allow a laboratory to upload results of an associated testing protocol.

### CROSS REFERENCE TO RELATED APPLICATIONS

This application claims the benefit of U.S. Provisional Application No.63/721602, filed Nov. 18, 2024, which is hereby incorporated by reference in its entirety.

### TECHNICAL FIELD

The present disclosure generally relates to the field of tracking, verification, and electronic documentation and authentication of formal processes. More particularly, the present disclosure relates to drug testing and monitoring, and more specifically to methods and systems for the tracking, verification, and authentication of multi-stage chain of custody for periodized drug tests and tracings, such as wearable drug patches.

### BACKGROUND

Wearable drug patches have become an essential tool in monitoring substance use and other physiological markers over extended periods of time. These patches, designed to collect sweat and other substances from the wearer, are typically worn for several days—anywhere from three (3) to fourteen (14) days—before being removed and sent to a laboratory for analysis. The collected specimens are analyzed for the presence of various substances, drugs, or biomarkers. Such wearable drug patches are often used in periodized or routine drug testing to aide in law enforcement, workplace compliance, or medical monitoring.

Traditionally, the process of tracking the application and removal of these patches has relied on a physical, paper-based, chain of custody, which is vulnerable to various inefficiencies. The current, market accepted, method involves the use of a carbon three-ply chain of custody form, which the wearer and technician manually fill out at the time of application and again at the time of removal in a single physical location. This method introduces several risks, including the potential loss of the physical form between application and removal, as well as errors in unverified, handwritten entries.

In this manual process, the wearer must also return to the same location for patch removal, i.e., the location at which the technician and the physical, paper-based, chain of custody form are housed, which poses logistical difficulties, particularly for wearers who undertake isolated or routine travel during the testing period. Furthermore, no electronic record for the respective device or testing protocol is created until the device reaches the selected laboratory, leaving openings in the chain of custody. Additionally, redundant data entry by manual means increases the likelihood of mistakes. These errors can lead to delayed or inaccurate test results, compromising the overall reliability of the process.

### BRIEF SUMMARY OF THE INVENTION

The present disclosure is directed to a method for tracking and verification of a periodized drug testing event using a wearable drug testing patch. The method is configured to, via a computing device having a processor and a non-transitory computer readable medium, execute the computer executable instructions embodied on the non-transitory computer readable medium and thereby execute the present method.

More particularly, the present disclosure further provides, according to one embodiment of the invention, a method for tracking the application and removal of a wearable drug testing patch including the following steps. First a unique client profile is created and stored on a user interface database written on the non-transitory computer readable medium, wherein the unique client profile is assigned a unique client identifier. Simultaneously with the creation of the unique client profile, a case record associated with the application of a drug patch having a patch serial number is created and stored on the user interface database written on the non-transitory computer readable medium, wherein the case record is assigned a unique case record identifier. Once the client profile and the case record are created and assigned the respective unique client identifier and the unique case identifier, the drug patch may be physically applied, at a first facility, to a wearer by a first technician wherein a patch application event is logged and recorded into the user interface database stored on the non-transitory computer readable medium. The patch application event includes, but is not limited to, capturing a first electronic signature from the wearer to verify patch application and wearer identity and assigning a first electronic signature identifier to the same and capturing a second electronic signature from the first technician to verify patch application and wearer identity and assigning a second electronic signature identifier to the same. Once the application event is logged and the first and second electronic signature are recorded, the unique client identifier, the unique case record identifier, the patch serial number, the first electronic signature identifier, and the second electronic signature identifier are linked in the user interface database and an application chain of custody document is compiled.

Following a pre-determined duration, i.e., the length of the testing period, the drug patch is physically removed from the wearer by a second technician, at a second facility, and a patch removal event is logged into the user interface database. The patch removal event includes, but is not limited to, capturing a third electronic signature from the wearer to verify patch removal and wearer identity and assigning a third electronic signature identifier to the same and capturing a fourth electronic signature from the second technician to verify patch application and wearer identity and assigning a fourth electronic signature identifier to the same. Once the removal event is logged and the third and fourth electronic signatures are recorded, a completed chain of custody document is compiled, wherein the chain of custody document comprises a unique barcode associated with each of the unique client identifier, the unique case record identifier, the patch serial number, the first electronic signature identifier, the second electronic signature identifier, the third electronic signature identifier, and the fourth electronic signature identifier. Once the completed chain of custody document is compiled the same is uploaded and recorded on the user interface database in connection with the pertinent case record and client profile.

Accordingly, once removed, the drug patch is placed in a closed receptable and sealed with a unique barcode sticker from a hard copy version of the completed chain of custody document. The sealed receptable and the completed chain of custody documents are then sent to a selected laboratory for testing and evaluation, with results of such testing uploaded and recorded on the user interface database and linked to and/or associated with the respective case record for access by the wearer, the client, a case manager, or another authorized party.

### BRIEF DESCRIPTION OF THE DRAWINGS

The embodiments set forth in the drawings are illustrative and exemplary in nature and not intended to limit the subject matter. The following detailed description of the illustrative embodiments can be understood when read in conjunction with the following drawings, where like structure is indicated with like reference numerals and in which:

FIG. 1. illustrates a flow diagram detailing the steps and sub-steps of the present method.

FIGS. 2A-2B further details the step of creating a unique client profile within the present method.

FIG. 3A-3B further details the step of opening a case record.

FIGS. 4A-4E further detail the sub-steps of applying the patch to the wearer and logging a patch application event of the present method.

FIGS. 5A-5E further details the sub-steps of requesting the verification of patch application and wearer identity via a first electronic signature from the wearer and assigning a first electronic signature identifier of the present method.

FIGS. 6A-6D further detail the steps of requesting the verification of patch application and wearer identity via a second electronic signature from the first technician and assigning a second electronic signature identifier; linking the unique client identifier, the unique case record identifier, the patch serial number, the first electronic signature identifier, and the second electronic signature identifier in the user interface database; and compiling an application chain of custody document for the linked unique client profile, the unique case record identifier, the patch serial number, the first electronic signature identifier, and the second electronic signature identifier and storing the same in the user interface database of the present method.

FIG. 7 further details the steps of notifying the wearer that the predetermined duration has lapsed and that the drug patch is due for removal; determining a current location of the wearer via a set of Global Positioning System (GPS) coordinates associated with the wearer's mobile electronic device; populating a list of removal locations that are geographically proximate to the wearer; and identifying and selecting a second facility for drug patch removal of the present method.

FIG. 8A-8B further details the step of accessing the case record associated with the application of the drug patch in the user interface database.

FIGS. 9A-9B further details the step of selecting the testing protocol for the drug patch and linking the selected testing protocol to the unique case record identifier in the user interface database.

FIGS. 10A-10C further details the step of logging a patch removal event in the user interface database, including, requesting the verification of patch removal and wearer identity via a third electronic signature from the wearer and assigning a third electronic signature identifier; requesting the verification of patch removal and wearer identity via a fourth electronic signature from a second technician and assigning a fourth electronic signature identifier; linking the third electronic signature identifier and the fourth electronic signature identifier to the case record in the user interface database; and compiling a completed chain of custody document for the case record, wherein the chain of custody document comprises a unique barcode associated with each of unique client identifier, the unique case record identifier, the patch serial number, the first electronic signature identifier, the second electronic signature identifier, the third electronic signature identifier and the fourth electronic signature identifier.

FIG. 11 further details the steps of receiving a result of the testing protocol from the laboratory; and linking the result of the testing protocol with the unique client identifier, the unique case record identifier, the patch serial number, and the chain of custody document in the user interface database, and storing the results in the user interface database; and providing access to the user interface database by the laboratory, the wearer, and a case manager to display and transmit the result of the selected testing protocol.

### DETAILED DESCRIPTION OF THE INVENTION

While the present disclosure may be described with respect to specific applications or industries, those skilled in the art will recognize the broader applicability of the disclosure.

Those having ordinary skill in the art will recognize the terms such as “a”, “an”, “the”, “at least one”, and “one or more” are used interchangeably to indicate that at least one of the items is present. A plurality of such items may be present unless the context clearly indicates otherwise. All numerical values of parameters (e.g., of quantities or conditions) in this specification, unless otherwise indicated expressly or clearly in view of the context, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, a disclosure of a range is to be understood as specifically disclosing all values and further divided ranges within the range.

The terms “comprising”, “including”, and “having” are inclusive and therefore specify the presence of stated features, steps, operations, elements, or components, but do not preclude the presence or addition of one or more other features, steps, operations, elements, or components. Orders of steps, processes, and operations may be altered when possible, and additional or alternative steps may be employed. As used in this specification, the term “or” includes any one and all combinations of the associated listed items. The term “any of” is understood to include any possible combination of referenced items, including “any one of” the referenced items. The term “any of” is understood to include any possible combination of referenced claims of the appended claims, including “any one of” the referenced claims.

Features shown in one figure may be combined with, substituted for, or modified by, features shown in any of the figures. Unless stated otherwise, no features, elements, or limitations are mutually exclusive of any other features, elements, or limitations. Furthermore, no features, elements, or limitations are absolutely required for operation. Any specific configurations shown in the figures are illustrative only and the specific configurations shown are not limiting of the claims or the description.

For consistency and convenience, directional adjectives are employed throughout this detailed description corresponding to the illustrated embodiments. Those having ordinary skill in the art will recognize that terms such as “above”, “below”, “upward”, “downward”, “top”, “bottom”, etc., may be used descriptively relative to the figures, without representing limitations on the scope of the invention, as defined by the claims. Any numerical designations, such as “first” or “second” are illustrative only and are not intended to limit the scope of the disclosure in any way.

Referring now to the drawings, wherein like reference numerals refer to like components throughout the several views, a system and method for drug testing and monitoring, and more specifically, to methods and systems for the tracking, verification, and authentication of multi-stage chain of custody for certain periodized drug tests and tracings, such as wearable drug patches are provided. While the system and method disclosed herein is generally described as being used for the tracing of wearable patches for the detection of drugs, it will be appreciated that the system and method described herein may be used in conjunction with the tracing of any number of applications where logging, verification, and verified electronic signatures are desired or necessary to maintain chain of custody.

As the demand for periodized or routine drug testing increases for mobile wearers, there is a growing need for an improved and verified system that automates the chain of custody, reduces human error, and allows for greater flexibility for wearers with respect to application, check-in, and removal locations. Moreover, an electronic system that tracks the patch from application to lab submission and links the same with a unique, verified, patient profile would provide a more secure and reliable method of documentation. As such, there is a need for a method of drug patch tracing and verification that incorporates electronic signatures, automated tracking, and remote patch inspection, providing a complete and secure chain of custody that ensures the integrity of the specimens and improves the overall efficiency and accuracy of the testing process.

In a general sense, the drug patch tracing, verification, and electronic system and method of the present disclosure are configured to track the application and removal of wearable patches, authenticate electronic signatures from wearers and technicians, and securely store chain of custody data for laboratory analysis and eventual delivery to a case manager or in some cases the court. Moreover, the system and method are designed to be flexible to allow application and removal of the respective patches by different technicians in different locations, while still maintaining the integrity of the testing protocol and chain of custody of the respective patch.

More particularly, the system comprises a non-transitory computer readable medium that stores a set of computer executable instructions; a user interface database written on and stored to the non-transitory computer readable medium, and at least one processor configured to execute the computer executable instructions embodied in the non-transitory computer readable medium, such that the non-transitory computer readable medium is configured to instruct the processor to execute the present method for tracking the application, removal, analysis, and delivery or reporting of results of a wearable drug testing patch, including logging wearer and technician signatures, generating chain of custody documents, and enabling data retrieval and analysis by a laboratory, and eventually a case manager.

The system may be deployed locally via a downloadable application or via a Software as a Service (SAAS) platform in a cloud computing environment accessible via a browser on any one of a number of computing devices, including, without limitation, a computer workstation, a desktop, notebook, laptop, a handheld computer, a mobile phone, a tablet, or some other computing device.

The system may include a non-transitory computer readable medium. The term non-transitory computer readable medium including any medium that participates in providing data (e.g., instructions) and facilitating the transfer of code, which may be read by a computer. Such a medium may take many forms accessible by a computer, including, but not limited to, non-volatile media, volatile media, etc. Non-volatile media include, for example, optical or magnetic disks and other persistent memory. Volatile media include dynamic random-access memory (DRAM), which typically constitutes a main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read, as well as cloud computing and networked versions of the same. The non-transitory computer readable medium stores or has written or embodied thereon a set of computer executable instructions that comprise the present method for tracking, verification, and authentication of multi-stage chain of custody for periodized drug tests and tracings, such as wearable drug patches.

A user interface database may also be written on or embodied in the non-transitory computer readable medium. The user interface module may be operative to implement a graphical user interface that can be stored in a mass storage device or in a cloud computing environment as executable software codes that are executed by the one or more computing devices. This and other modules can include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

Databases or data stores described herein may include various kinds of data, including a hierarchical database, a set of files in a files system, an application database in a proprietary format, a relational database management system (RDBMS), a nonrelational database management system, a look-up table, etc. Each database or data store is generally included with a computing device employing a computer operating system and may be accessed via a network in any one or more variety of manners.

The user interface database is configured to store a compilation of patch application data, chain of custody records, wearer information, technician information, agency information, patch condition images and videos, electronic signatures and screening details. For example, the user interface database may be populated with patient or wearer profile information, technician-entered information such as drug patch serial number, patch application location and time stamps, patch application and removal timestamps, digital signatures obtained from the wearer and technician during the application and removal process, selected drug testing protocols, and laboratory test scheduling and result delivery details. The drug patch tracking database may be populated with wearer-entered information such as images or videos of the patch, confirmation of patch condition, and digital signatures during the application and removal processes, as further detailed herein below with respect to the present method 100.

The system may further comprise at least one processor configured to execute the computer executable instructions embodied on the non-transitory computer readable medium. Computer-executable instructions may be compiled or interpreted from computer programs, software code, or algorithms created using a variety of programming languages and/or technologies, including, without limitation, and either alone or in combination, Java™, C, C++, Visual Basic, Java Script, Perl, html, etc. In general, a processor (e.g., a microprocessor) receives instructions, e.g., from a memory, a computer-readable medium, etc., and executes these instructions, thereby performing one or more processes, including one or more of the processes described within the present method. Such instructions and other data may be stored and transmitted using a variety of computer-readable media. It is appreciated that software modules can be callable from other modules or from themselves, and/or can be invoked in response to detected events or interrupts. The modules, computer executable instructions, and/or computing device functionality described herein are preferably implemented as software modules, but can be represented in hardware or firmware. Generally, the modules, computer executable instructions, and/or computing device functionality described herein refer to logical modules that can be combined with other modules or divided into sub-modules despite their physical organization or storage.

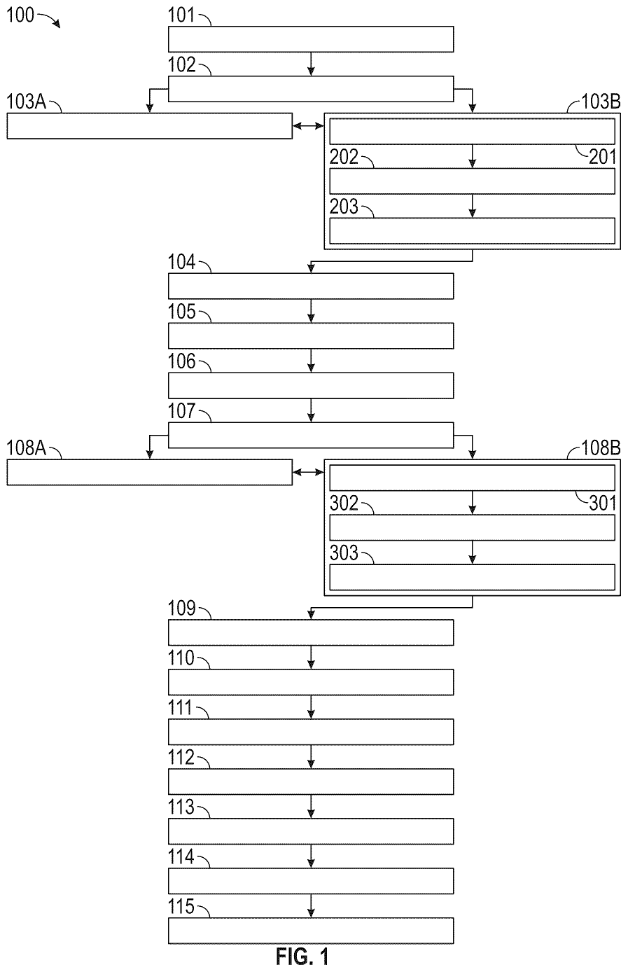

Referring now to the drawings, exemplary methods for tracking, verification, and authentication of multi-stage chain of custody for periodized drug tests and tracings, such as wearable drug patches are provided. As detailed herein, the at least one processor is configured to execute the computer executable instructions embodied in the non-transitory computer readable medium, such that the non-transitory computer readable medium is configured to instruct the processor to access the user interface database and execute the present method 100. The present method 100 for drug patch tracing, verification, and electronic signature management is detailed further in FIG. 1 and comprises several, namely, steps 101-115.

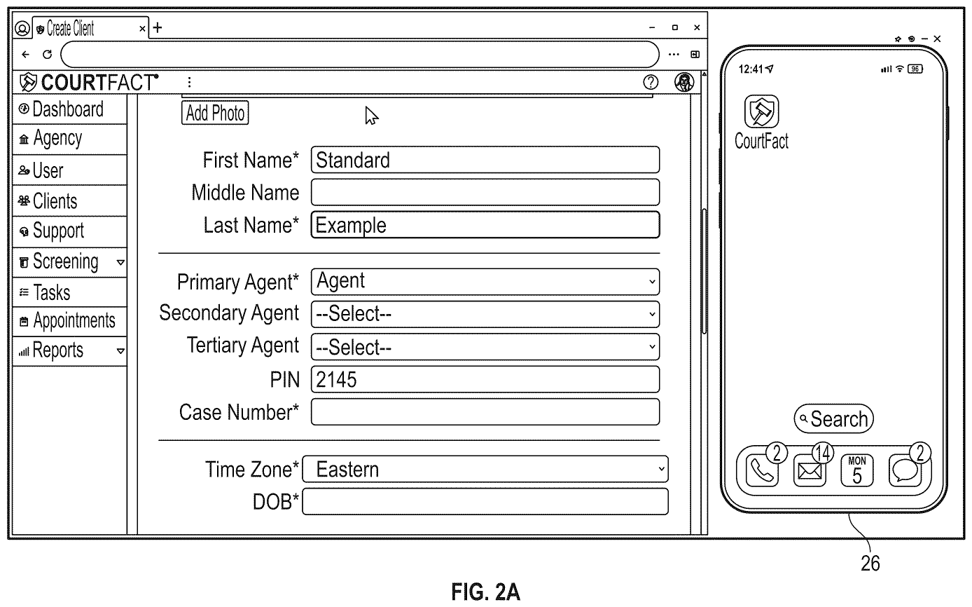

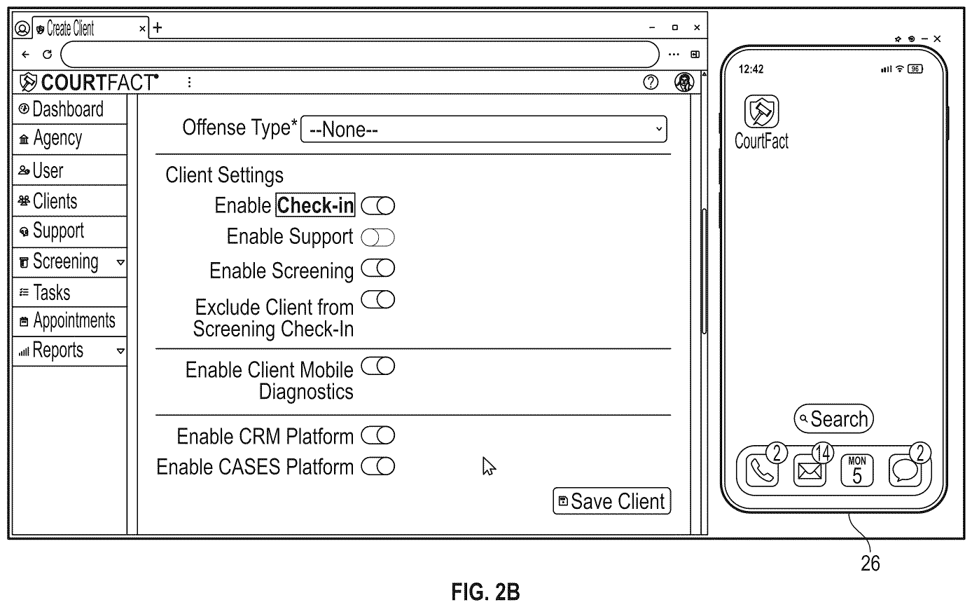

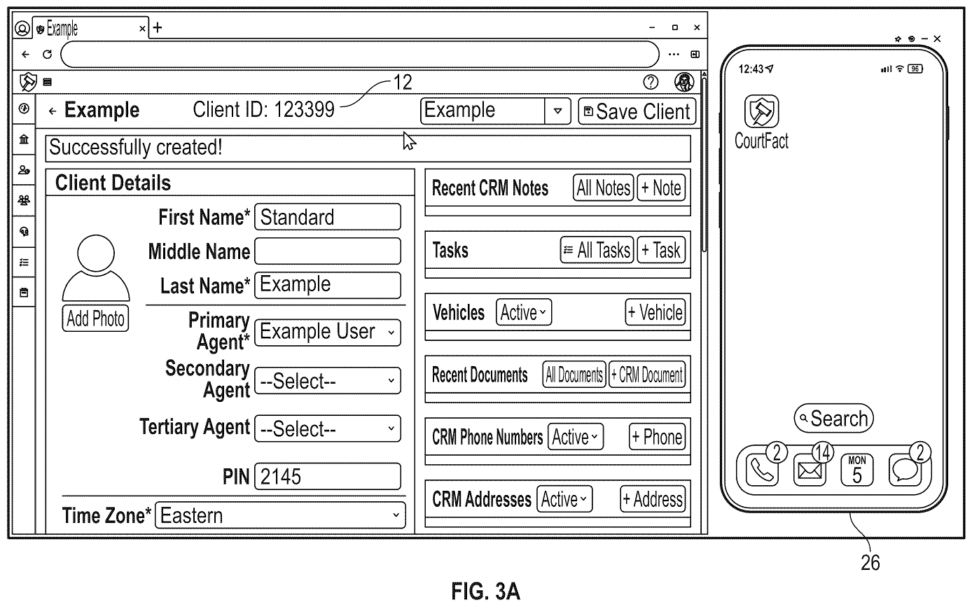

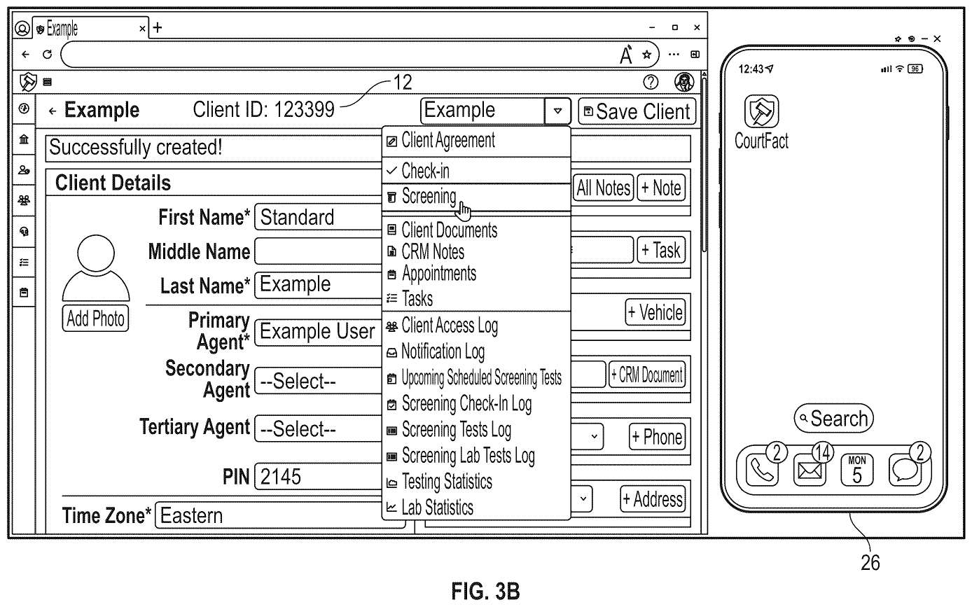

Referring to FIG. 1 and FIGS. 2A-2B, at step 101, agency and wearer information are loaded into the user interface database, via an input interface provided through a user-facing module, to create a unique client profile. The unique client profile is assigned a unique client identifier 12 (FIGS. 3A-3B) and stored on the user interface database written on the on-transitory computer readable medium.

As described herein, agency may refer to the organization or entity responsible for overseeing or administrating the program in which the wearer is participating. The agency may be a government entity, such as a law enforcement agency, court system, probation department, or a private organization, rehabilitation facility, healthcare provider, or medical institution. The agency may charge a case manager, such as a parole officer or other agent, with the oversight or administration of the respective drug testing protocol of the wearer. Agency information may include, but is not limited to, the agency's name, the agency's address, the agency's contact details including phone numbers and/or email addresses for the case manager, and other relevant information.

As described herein, the wearer refers to the individual within the drug testing program and to whom a drug testing patch 14 is applied. Wearer information may include but is not limited to, the wearer's name, the wearer's photo or another recordation of likeness, date of birth, gender identity, program start date, program end date, state and county of court, court jurisdiction, associated agency and case manager, and other relevant identifiers or enrollment information. Upon entry of the wearer information and agency information, the system generates the unique client identifier 12. The unique client identifier 12 may be a distinct string of alphanumeric characters or numbers associated with the wearer and the unique client profile. This unique client identifier 12 is critical for linking the unique client profile to all subsequent interactions, including patch 14 application and patch 14 removal and resulting laboratory results. The agency information and the unique client profile are organized and stored in the user interface database for future reference, retrieval, and linking of additional process information.

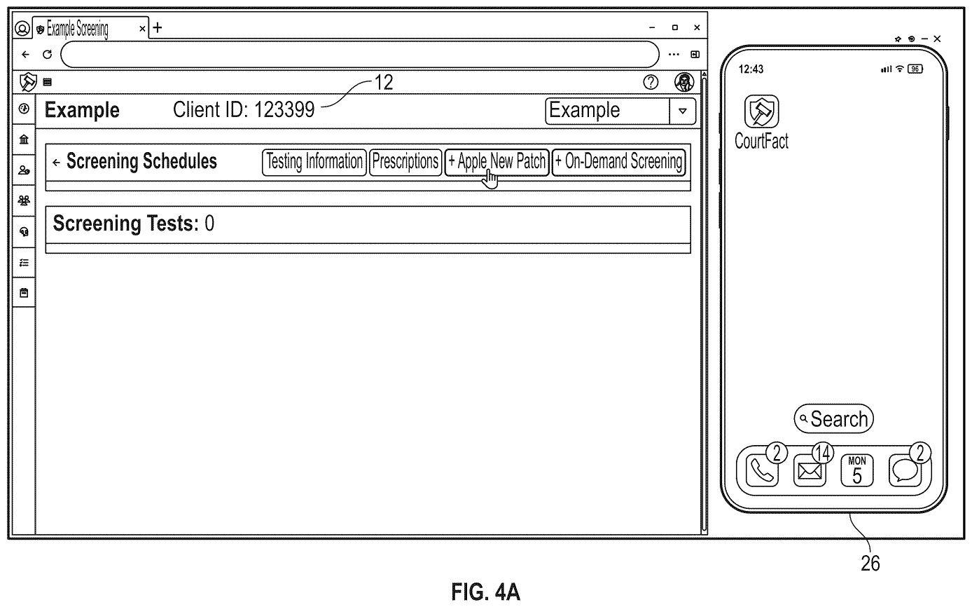

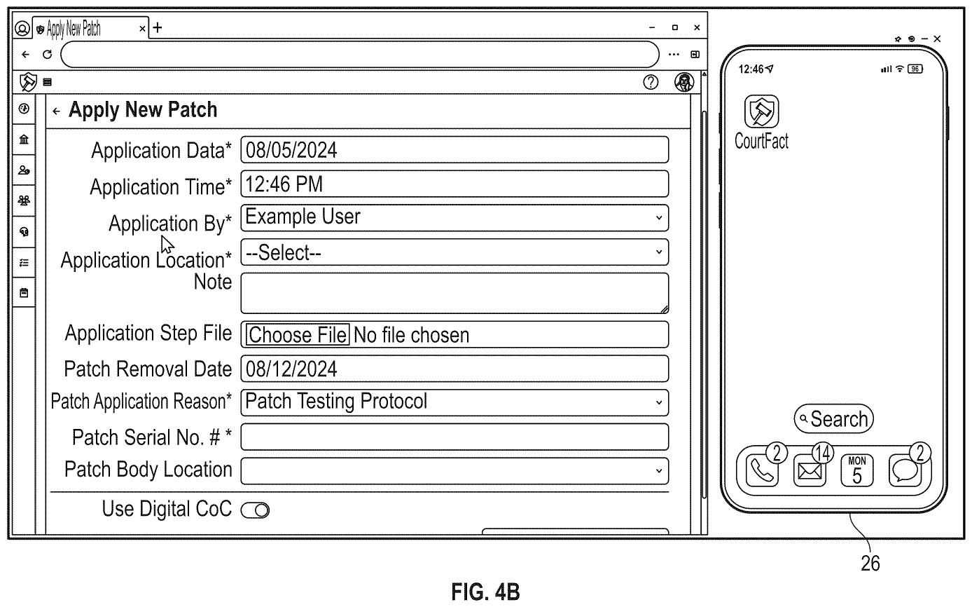

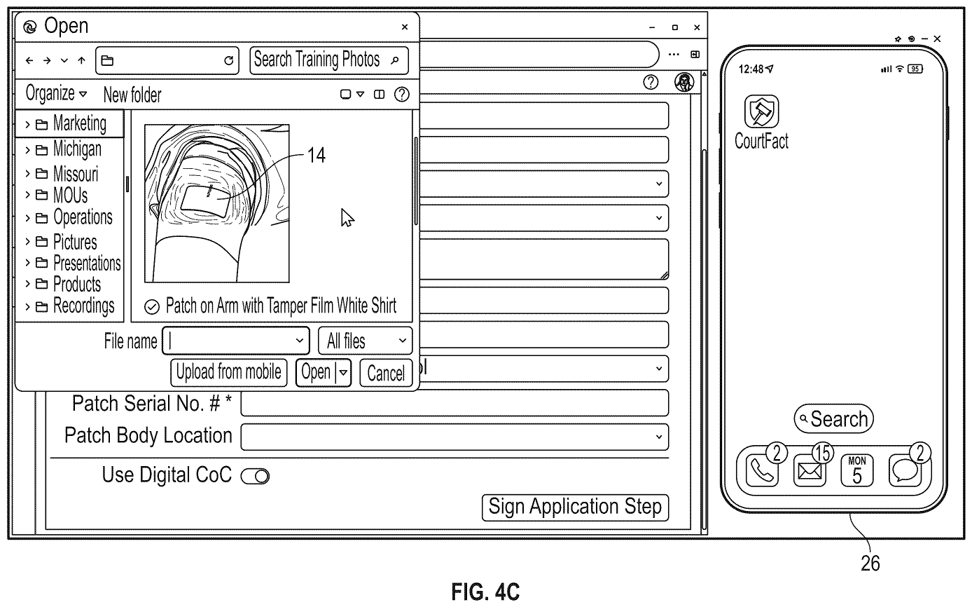

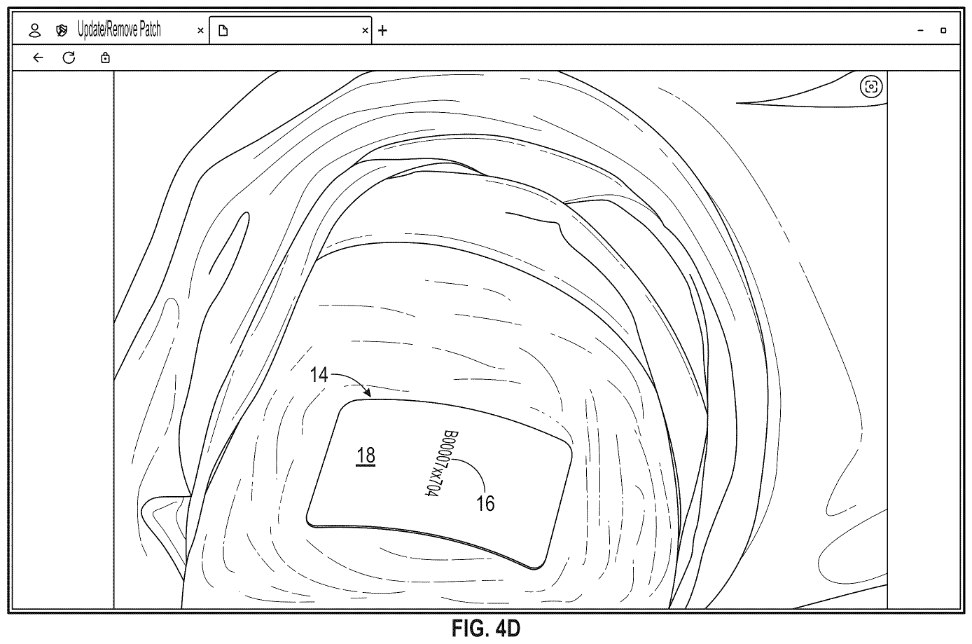

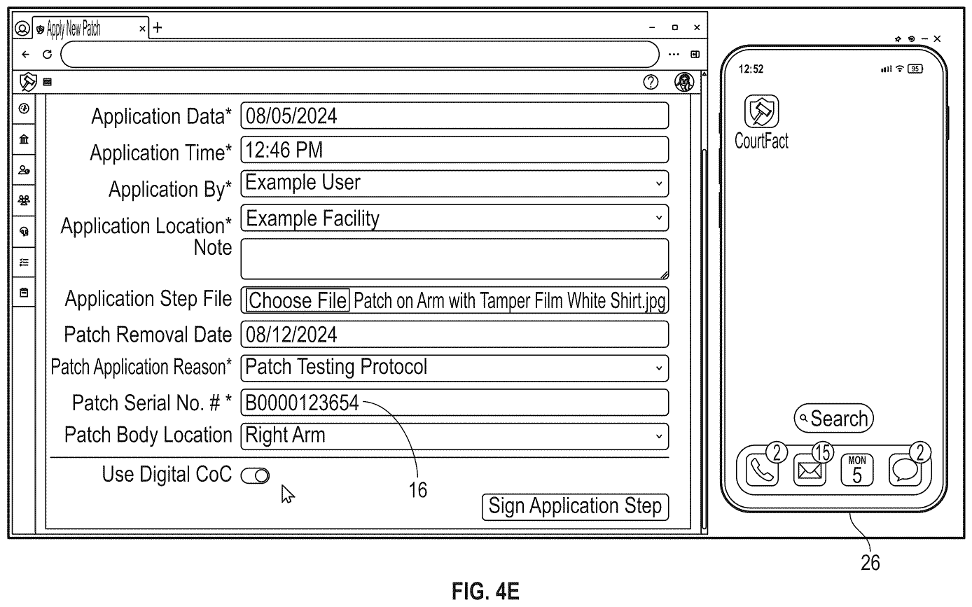

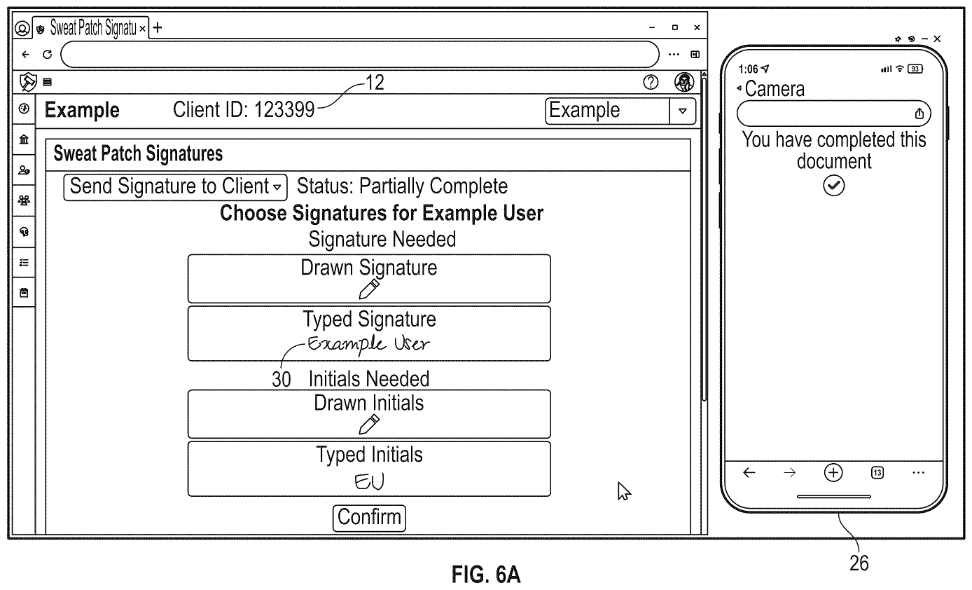

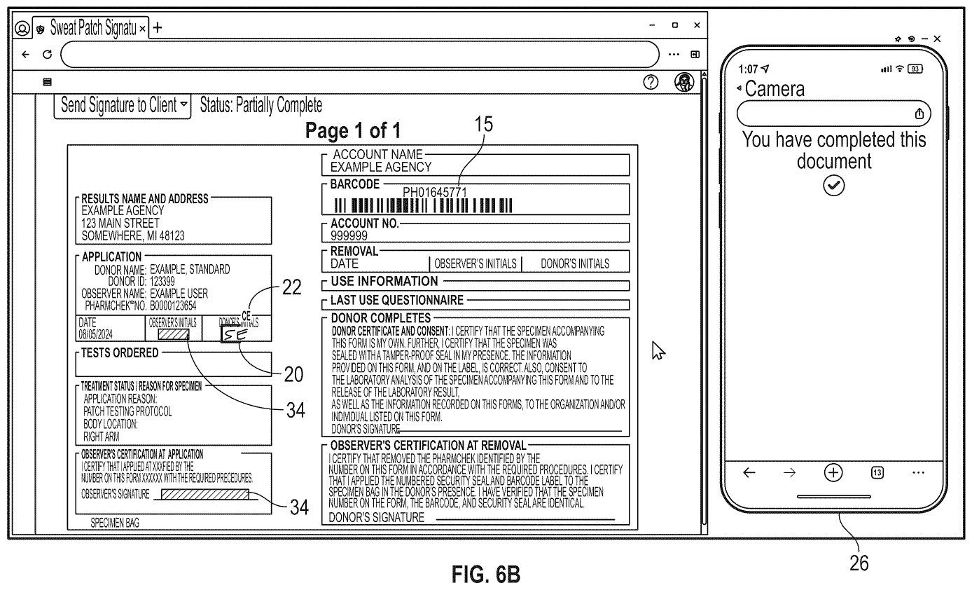

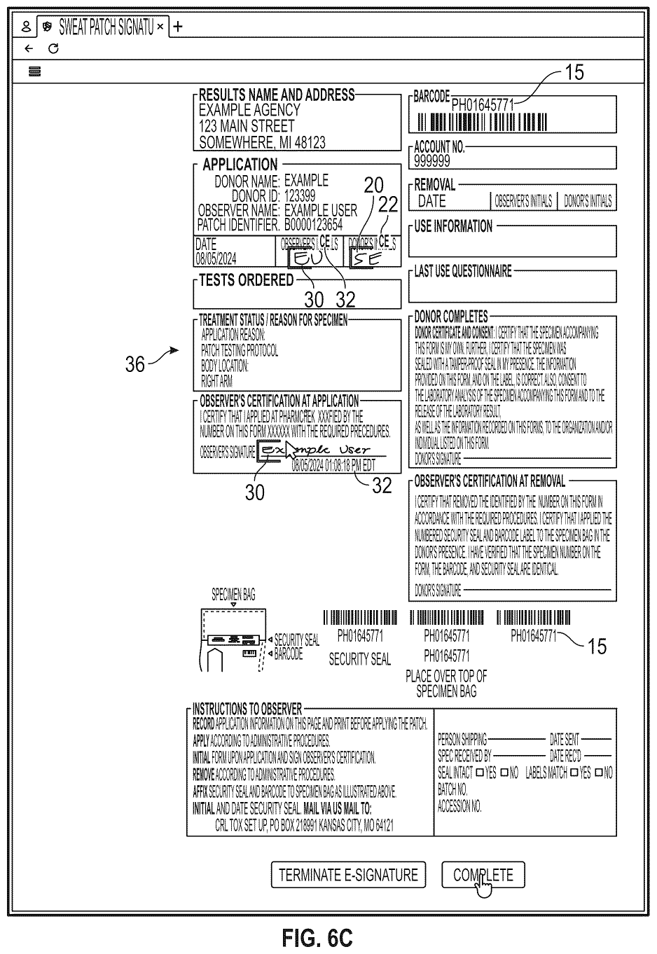

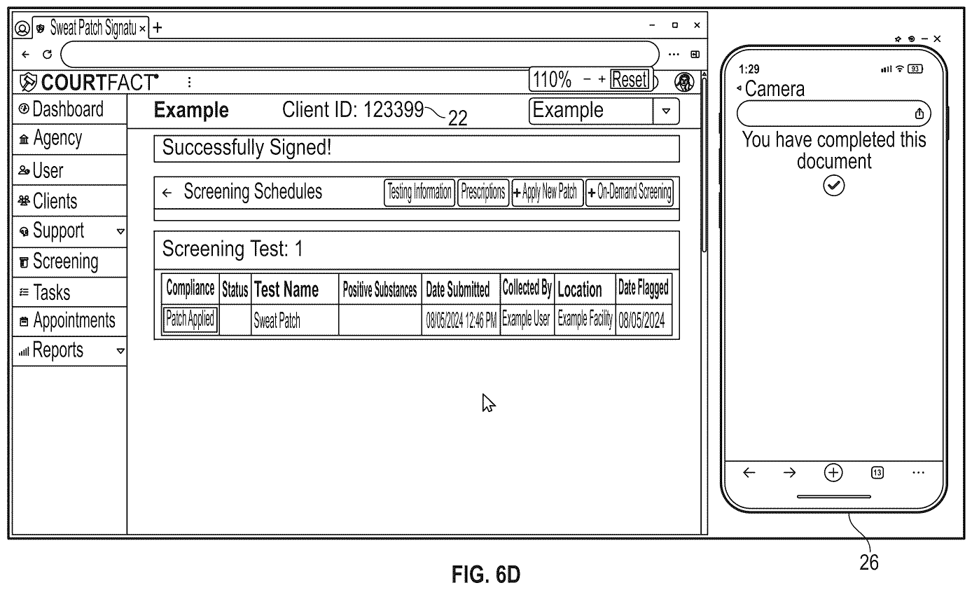

Referring to FIG. 1 and FIGS. 3A-3B, next, at step 102, a case record, associated with the application of a drug patch 14 having a patch serial number 16 (FIGS. 4D and 4E), is opened and stored in the user interface database. The case record is assigned a unique case record identifier 15 (FIGS. 6B, 6C). The case record identifier 15 may be a distinct string of alphanumeric characters or numbers. The caser record identifier 15 is linked on the user interface database with the unique client identifier 12 to ensure that the patch 14 and its associated patch serial number 16 (FIGS. 4D and 4E) and other data are tied to the specific wearer throughout the monitoring process.

Referring to FIG. 1, next at step 103A the drug patch 14 having the drug patch serial number 16 is applied to a wearer by a first technician at a first facility. The drug testing patch 14 may be designed to be worn on and in contact with the wearer's skin in a predetermined location for a predetermined duration of wear. For example, the drug testing patch 14 may be adhesively applied to the wearer's left or right arm (FIG. 4D), left or right leg, left or right upper back, left or right lower back, or other body location for the predetermined duration of wear. Each drug patch 14 is designed for wear over a predetermined duration, which is driven by the respective laboratory tests to be completed upon removal of the patch 14 as detailed herein below. In some example embodiments, the predetermined duration is from about three (3) days to about fourteen (14) days. In one example, the predetermined duration is about seven (7) days.

The drug testing patch 14 may contain an absorbent member 18 (FIG. 4D) that may capture the wearer's sweat or other byproducts excreted through the skin over the course of the predetermined duration of wear, thereby allowing for the detection of various drug metabolites. The drug testing patch 14 may aid in the detection of various substances, including but not limited to, amphetamine, methamphetamine, cocaine, opiates, marijuana, fentanyl, norfentanyl, phencyclidine, hydrocodone, oxymorphone, oxycodone, benzodiazepines, methadone, buprenorphine, and norbuprenorphine. The drug testing patch 14 may be any known drug testing patch 14 to those in the relevant field. Such suitable drug testing patches 14 are commercially available from, for example, manufacturer PharmChem, namely the PharmChek® Drugs of Abuse Sweat Patch. Such drug patches 14 are provided to be tamper evident, such that it will be clear to an ordinary observer, but surely an application or removal technician if the patch 14 has been removed, tampered with, or otherwise compromised by the wearer.

Referring still to FIG. 1 and FIGS. 4A-4E, simultaneously, with the physical application of the drug patch 14 to the wearer in step 103A, at step 103B a patch application event is logged into the user interface database, as shown in FIGS. 4A-4E.

Once initiated, as shown in FIG. 4A, the patch application event may be automatically dated and timestamped by the system (FIG. 4B). The first technician may then input additional information including, but not limited to, the name of the first technician applying the drug testing patch 14 to the wearer, information related to the first facility at which the drug testing patch 14 is being applied, the predetermined duration and anticipated patch removal date, the reason for patch 14 application, the patch serial number 16 (FIG. 4C), and a description of the bodily location at which the patch 14 was applied to the wearer, e.g., the wearer's left or right arm (FIG. 4D), left or right leg, left or right upper back, left or right lower back, or other body location.

In one example embodiment, as shown in FIG. 4C, the first technician may capture, via an independent capturing device, such as a camera or the like, at least one image of the drug patch 14 as applied to the wearer at the time of application, and upload or store the at least one image to the user interface database with a date stamp and time stamp related to the image. The at least one image is configured to confirm the bodily location of placement of the patch 14 upon the wearer and the state in which the patch 14 was applied, as well as the patch serial number 16. The image may also include a capture of the likeness or face of the wearer, such that the identity of the wearer may be visually verified at the time of patch 14 application and patch 14 removal.

Step 103B, is further defined by several sub-steps detailed as steps 201-203 in FIG. 1. Once the relevant case record information is recorded in the user interface database, the system requests a variety of confirmations from the wearer and the first technician in the form of electronic signatures to verify the patch 14 application and the wearer's identity. The electronic signature or verification may also serve as an acknowledgment by the wearer that the patch has been applied correctly and/or that the wearer understands any terms and conditions associated with the monitoring program, as well as an acknowledgement and confirmation by the first technician that he/she applied the subject patch 14, having the identified serial number 16, to the subject wearer, in the first location specified in the case record, as detailed in the recorded image.

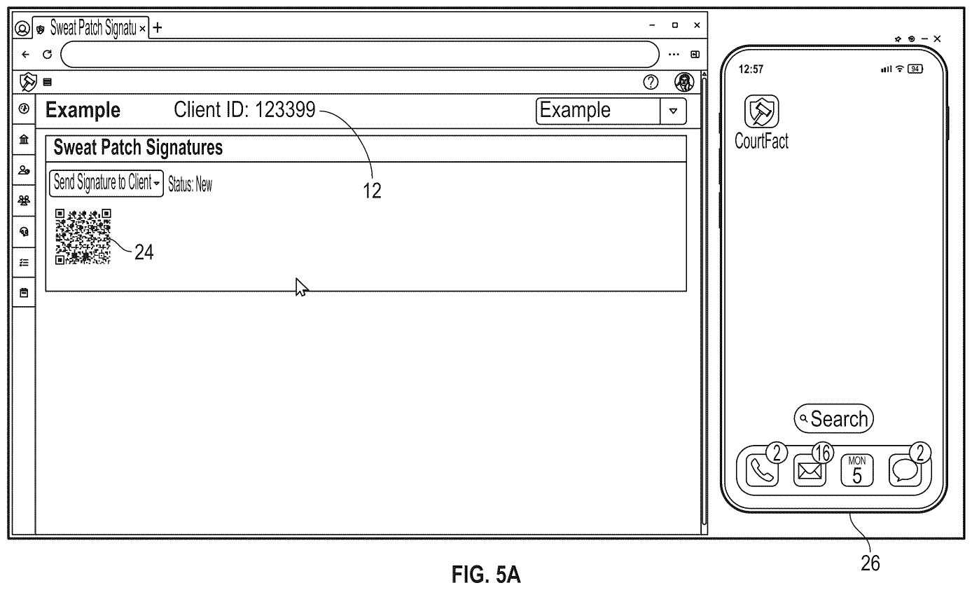

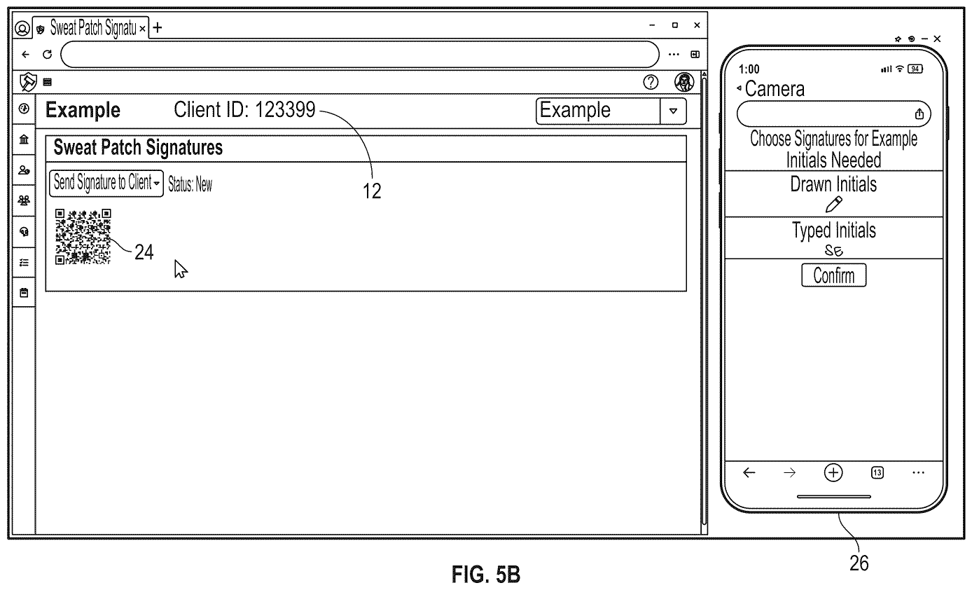

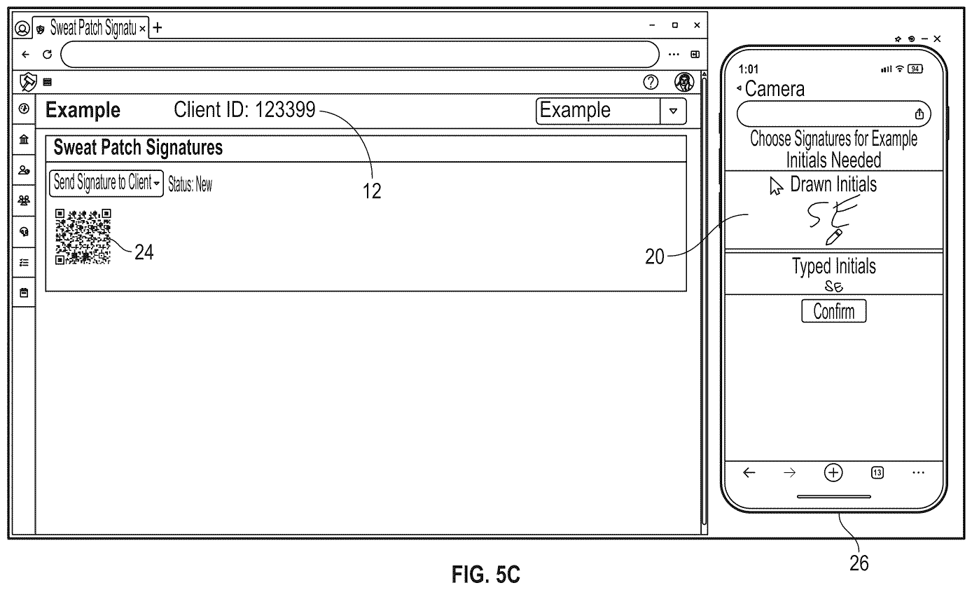

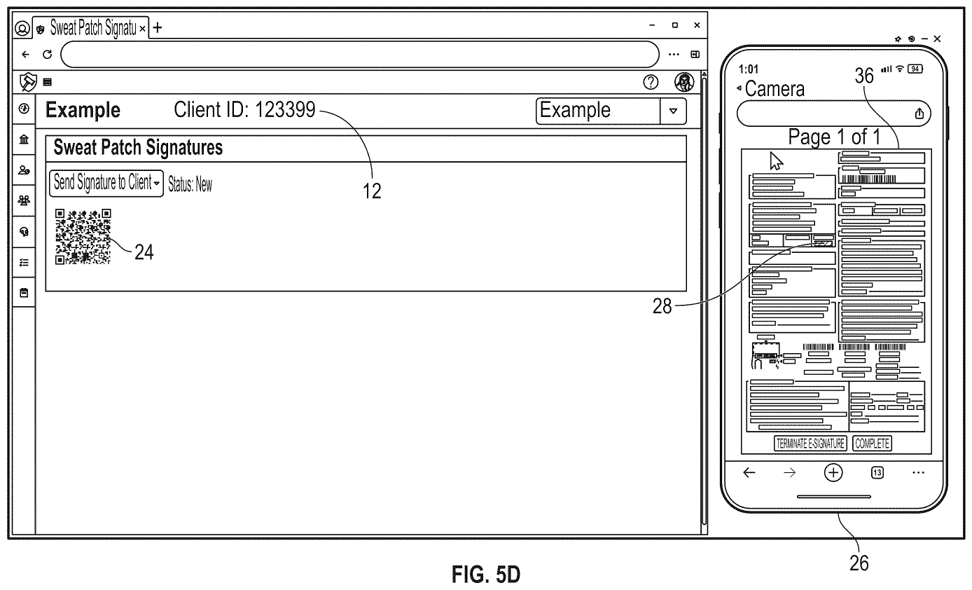

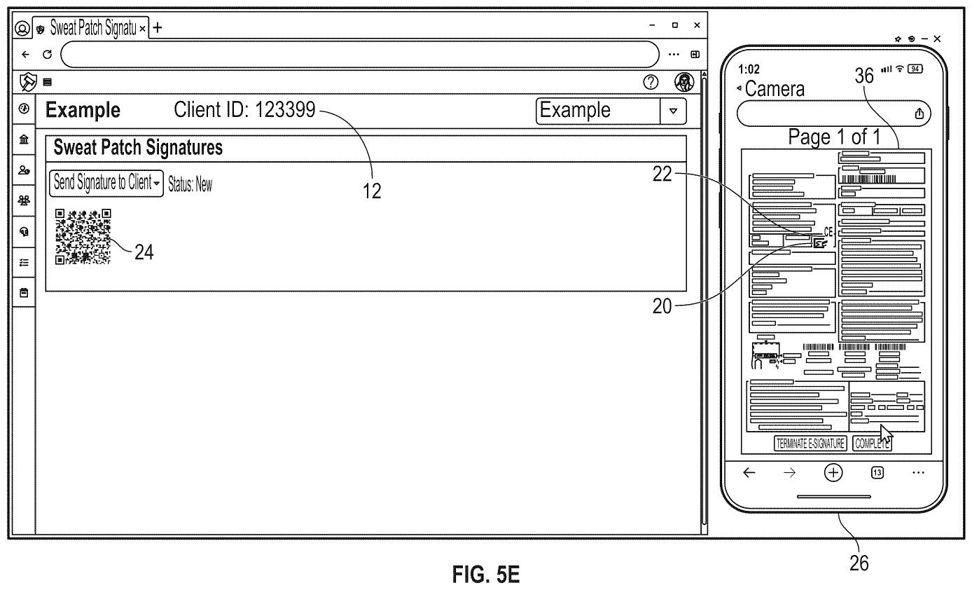

Referring to FIGS. 5A-5E, at step 201, the system requests the verification of patch application and wearer identity via a first electronic signature 20 from the wearer and assigns a first electronic signature identifier 22 to the first electronic signature. More particularly, the system initiates a unique one-time-use-code 24 and transmits the same to the wearer, such that the wearer can access the one-time-use-code 24 through various methods including, but not limited to, a scannable QR code displayed on the technician's or wearer's device 26, a text message link sent to the wearer's phone, or an email link sent to the wearer (FIG. 5A). Once the wearer accesses the one-time-use code 24, the wearer is presented with the option to verify patch application and/or his/her identity via use of the system-generated initials or physically sign with their finger or stylus on a touchscreen-enabled device (FIG. 5B).

Upon collecting the first electronic signature 20 from the wearer, the system begins the compilation of an initial application chain of custody document (FIG. 5D) and displays the same to the wearer's device 26 for signature and confirmation to signature categories 28, wherein the wearer may review the content and apply their initials/electronic signature using a highlighted visual cue to ensure the correct signature placement and thereby authenticate and authorize the electronic signature, at which point the wearer's initials are verified and entered into the initial application chain of custody document 36 (FIG. 5E). After reviewing and confirming the details, and authorization of the electronic signature 22, the wearer clicks a complete button to finalize their portion of the electronic signature process and generate a first electronic signature identifier 22 securely stored in the user interface database and linked with unique client identifier 12, the unique case record identifier 15, and the patch serial number 16.

Referring to FIGS. 6A-6C, at step 202, the system requests the verification of patch application and wearer identity via a second electronic signature 30 from the first technician and assigning a second electronic signature identifier 32. More particularly, after the wearer has provided the first electronic signature 22, the system prompts the first technician to verify the patch application and wearer identity via a second electronic signature 30. The second electronic signature 30 may serve as an acknowledgment that the patch 14 has been properly applied and/or that the first technician is verifying the details of the patch application. The electronic signature process for the first technician is initiated by generating a unique one-time-use code, which can be accessed by the technician through various methods, including but not limited to, a scannable QR code displayed on the first technician's device, a text message link sent to the first technician's phone, or an email link sent to the first technician. Once the first technician accesses the electronic signature via a common web browser, the first technician is then prompted to use the system-generated initials and signature or physically sign with their finger or stylus on a touchscreen-enabled device (FIG. 6A).

Upon collecting the electronic signature from the first technician, at step 203, the system applies the second electronic signature identifier 32 to the initial application chain of custody document 36 (FIG. 6B-6C) and displays the same to the first technician for signature and confirmation to signature categories 34, wherein the first technician may review the content and apply their initials/electronic signature using a highlighted visual cue to ensure the correct signature placement and thereby authenticate and authorize the second electronic signature 30, at which point the first technician's initials are verified and entered into the initial application chain of custody document 36 (FIG. 6B-6C). After reviewing and confirming the details, and authorization of the second electronic signature 30, the first technician clicks a complete button to finalize their portion of the electronic signature process and generate a second electronic signature identifier 32 to be securely stored in the user interface database and linked with the fist electronic signature identifier 22, the unique client identifier 12, the unique case record identifier 15, and the patch serial number 16.

Referring back to FIG. 1, following completion of the patch application event and successful capture of the system-authenticated signatures at steps 103A and 103B, at step 104, the system links each of the unique client identifier 12, the unique case record identifier 15, the patch serial number 16, the first electronic signature identifier 22, and the second electronic signature identifier 32 in the user interface database. At step 105, and shown in FIG. 6C, the system compiles an application chain of custody document 36 for the linked unique client profile 12, the unique case record identifier 15, the patch serial number 16, the first electronic signature identifier 22, and the second electronic signature identifier 32 and stores the same in the user interface database as shown in FIG. 6D, such that details and information associated therewith are stored and available for access at any time.

At step 106, once the system compiles an application chain of custody document 36 the system may then optionally notify the case manager, such as a parole officer or other agent, via a push notification generated from the user interface database and transmitted to the agent's mobile electronic device, that the drug patch 14 has been applied and that the predetermined duration associated therewith has started to toll. The system may also provide the case manager or agent with access to the details and information associated therewith including the anticipated removal date for the respective patch 14.

Optionally, during the predetermined duration of wear, the system may provide for remote intermittent inspection of the worn drug testing patch 14. In such instances, the system may notify the wearer, via a push notification generated from the user interface database and transmitted to a wearer mobile electronic device 26, that an intermittent inspection of the patch 14 is required. In one example embodiment, the wearer may capture, via an independent capturing device, such as a camera or the like, at least one image of the drug patch 14 as applied to the wearer at the time of intermittent inspection. The wearer may then upload or store the at least one image to the user interface database with a date stamp and time stamp related to the image. The at least one image is configured to confirm the location of placement of the patch 14 upon the wearer, the state of the patch 14 at the time of intermittent inspection, as well as the patch serial number 16. The image may also include a capture of the likeness or face of the wearer, such that the identity of the wearer may be visually verified.

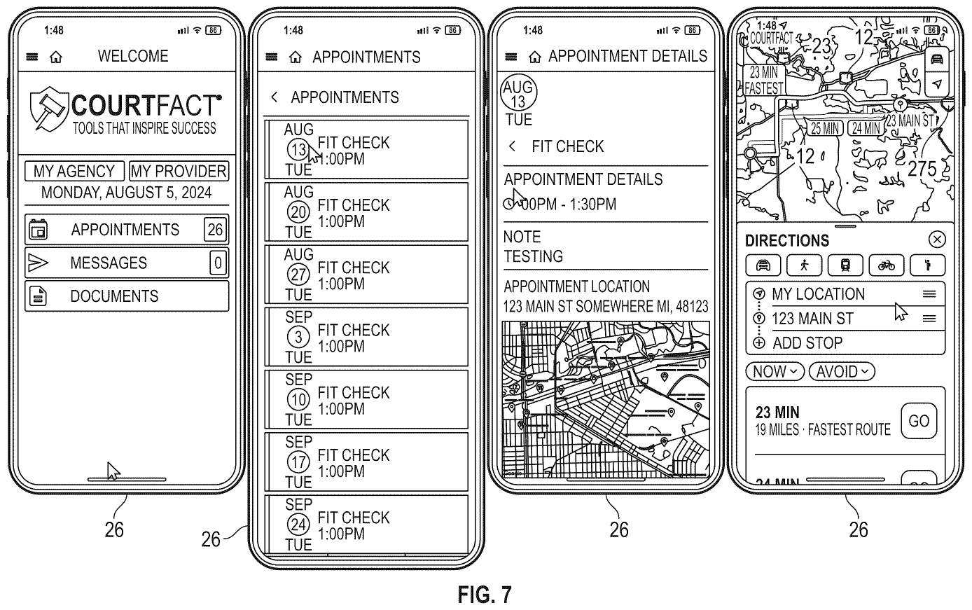

At step 107, and as shown in FIG. 7, following the end of the predetermined duration of wear, the system may notify the wearer, via a push notification generated from the user interface database and transmitted to a wearer mobile electronic device, that the predetermined duration has lapsed and that the drug patch 14 is due for removal. More particularly, the system may determine a current location of the wearer via a set of Global Positioning System (GPS) coordinates associated with the wearer's mobile electronic device and populate a list of removal locations that are geographically proximate to the wearer, such that the wearer may identify and select a second facility for drug patch removal from the list of removal locations that are geographically proximate to the wearer.

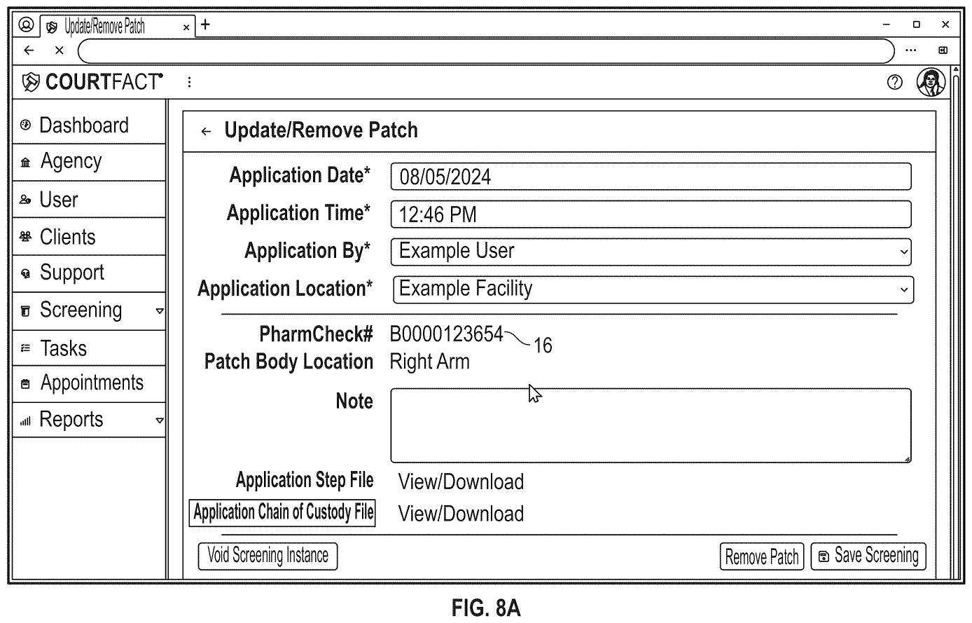

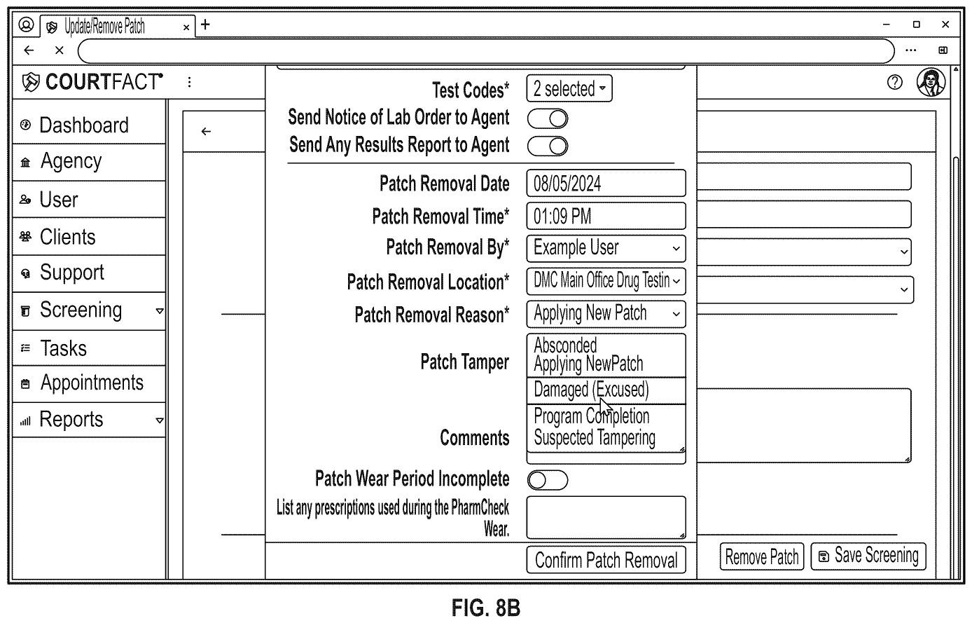

Following the pre-determined duration, i.e., the length of the testing period, the drug patch 14 is physically removed from the wearer by a second technician, at a second facility, at step 108A and placed into a closed receptacle. Simultaneously, with the physical removal of the drug patch 14 from the wearer in step 108A, at step 108B a patch removal event is logged into the user interface database, as shown in FIGS. 8A-8B. While it is contemplated that physical removal of the patch 14 occurs simultaneously with completion of the patch removal event in the system, it is also contemplated that physical removal of the patch 14 may be initiated before or after the patch removal event is logged into the system.

It is a distinct object of the present disclosure that the first facility may be the same as the second facility or the first facility may be different that the second facility. Analogously, the first technician may be the same as the second technician and the second technician or the first technician may be a different technician at the same or a different facility. Accordingly, the system and method are designed to be flexible to allow application and removal of the respective patches 14 by different technicians in different locations, while still maintaining the integrity of the testing protocol and chain of custody of the respective patch 14. This allows flexibility to the wearer to attend to the proper protocols regardless of relevant travel or other obligations.

The patch removal event, at step 108B and as detailed in FIGS. 8A-8B, is further defined by several sub-steps detailed as steps 301-303 in FIG. 1. At sub-step 301, the second technician accesses the case record 15 associated with the application to the drug patch 14, such that the patch removal event may be automatically dated and timestamped by the system. The second technician may further, at sub-step 302, input additional confirming information into the case record 15 (FIG. 8A), including, but not limited to, the name of the second technician removing the drug testing patch 14 from the wearer, information related to the second facility at which the drug testing patch 14 is being removed, the patch removal date, the reason for patch 14 removal, the patch serial number 16, and a description of the bodily location at which the patch 14 was applied to the wearer prior to removal, e.g., the wearer's left or right arm (FIG. 4D), left or right leg, left or right upper back, left or right lower back, or other body location.

In some instances, the second technician may capture, via an independent capturing device, such as a camera or the like, at least one image of the drug patch 14 as applied to the wearer at the time of removal, and upload or store the at least one image to the user interface database with a date stamp and time stamp related to the image. The at least one image is configured to confirm the location of placement of the patch 14 upon the wearer and the state of the patch 14 at the time of removal, i.e., whether the patch was tampered with, as well as the patch serial number 16. The image may also include a capture of the likeness or face of the wearer, such that the identity of the wearer may be visually verified.

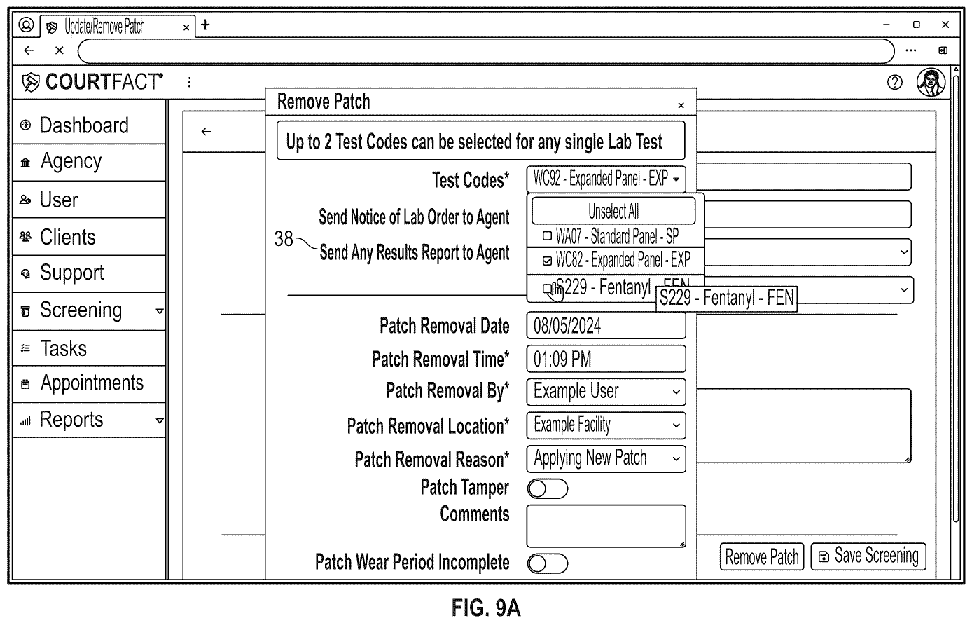

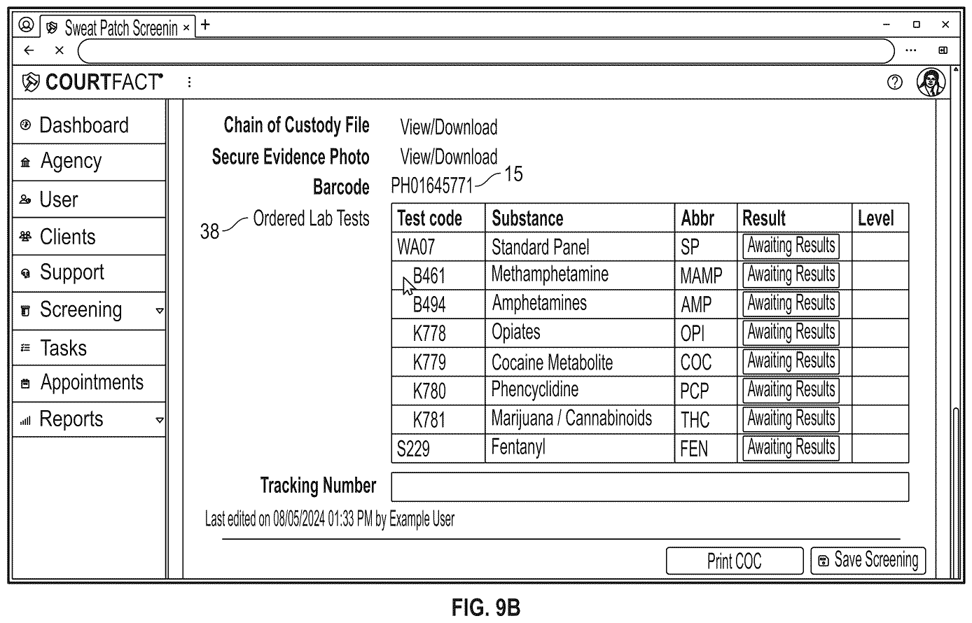

At step 303, and as shown in FIG. 9A, the second technician selects the testing protocol 38 for the drug patch 14 and links the selected testing protocol 38 to the unique case record identifier 15 in the user interface database. The selected testing protocol 38 comprises the lab tests to be performed on the patch 14. As shown in FIG. 9B, the testing protocol 38 protocol is configured to include groupings of tests associated with at least one of amphetamine, methamphetamine, cocaine, opiates, marijuana, fentanyl, norfentanyl, phencyclidine, hydrocodone, oxymorphone, oxycodone, benzodiazepines, methadone, buprenorphine, and norbuprenorphine.

Once all necessary information is entered in the case record by the second technician at sub-step 302 and the testing protocol 38 is selected by the second technician at sub-step 303, the system requests a variety of confirmations from the wearer and the second technician in the form of electronic signatures to verify the patch 14 removal and the wearer's identity. The electronic signature or verification may also serve as an acknowledgment by the wearer that the patch 14 has been removed, as well as an acknowledgement and confirmation by the second technician that he/she removed the subject patch 14 having a noted serial number 16 to the subject wearer, at the second facility detailed in the case record, as detailed in the recorded image.

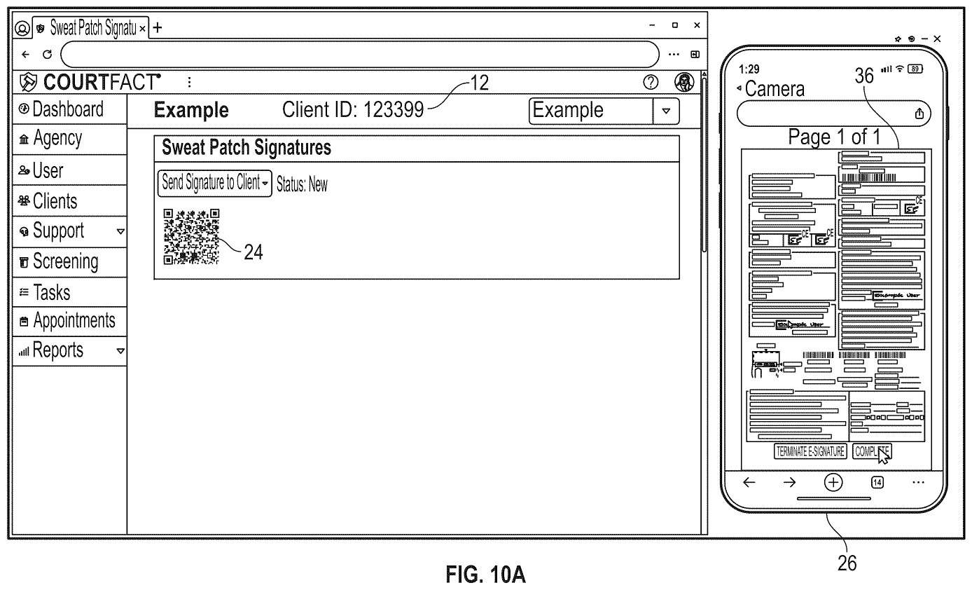

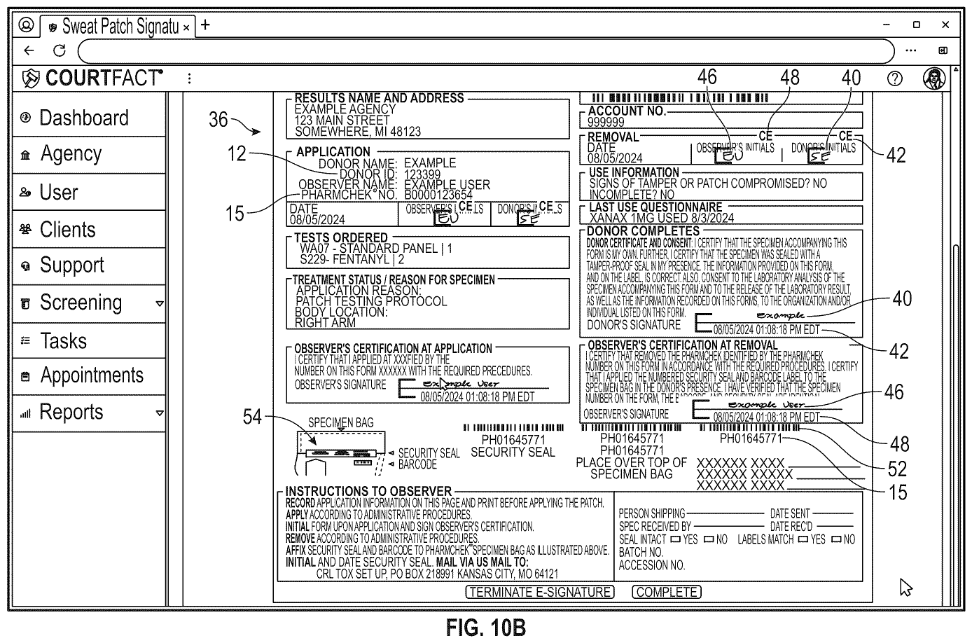

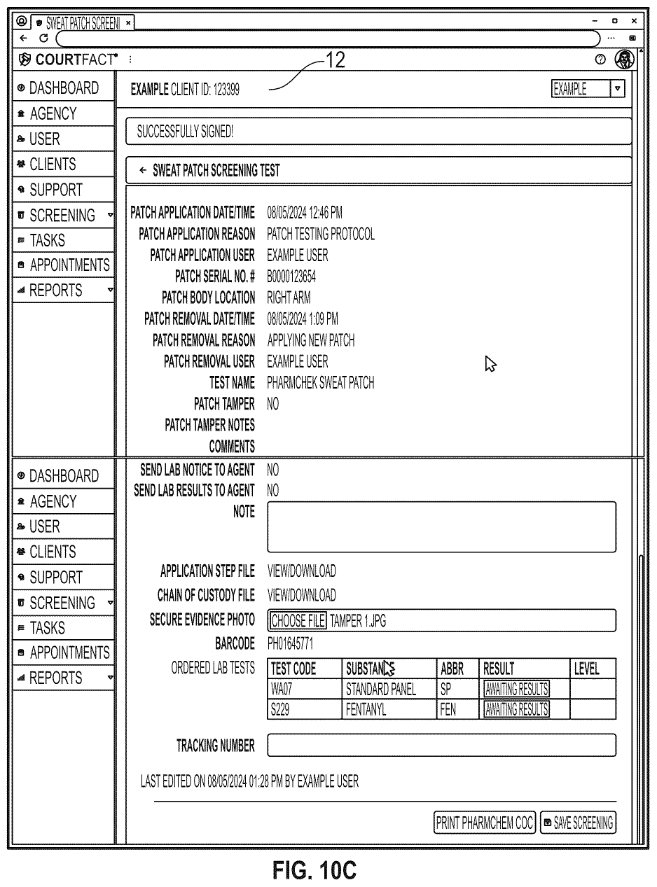

Referring to FIGS. 10A-10C, at step 109, the system requests the verification of patch removal and wearer identity via a third electronic signature 40 from the wearer and assigning a third electronic signature identifier 42. More particularly, the system initiates a unique one-time-use-code 24 and transmits the same to the wearer, such that the wearer can access the one-time-use-code 24 through various methods including, but not limited to, a scannable QR code displayed on the technician's or wearer's device 26, a text message link sent to the wearer's phone, or an email link sent to the wearer (FIG. 10A). Once the wearer accesses the one-time-use code 24, the wearer is presented with the option to verify patch removal and/or his/her identity via use of the system-generated signature or credentials.

Upon collecting the electronic signature from the wearer, the system accesses the initial application chain of custody document 36 and displays the same to the wearer's device 26 for signature and confirmation, wherein the wearer may review the content and apply their electronic signature 40 using a highlighted visual cue to ensure the correct signature placement and thereby authenticate and authorize the electronic signature 40, at which point the wearer's signature is verified and entered into the chain of custody document 36 (FIGS. 10A-10B). After reviewing and confirming the details, and authorization of the third electronic signature 40, the wearer clicks a complete button to finalize their portion of the electronic signature process and generate a third electronic signature identifier 42 securely stored in the user interface database and linked with unique client identifier 12, the unique case record identifier 15, and the patch serial number 16.

At step 110, the system requests the verification of patch removal and wearer identity via a fourth electronic signature 46 from the second technician and assigning a fourth electronic signature identifier 48. More particularly, after the wearer has provided the third electronic signature 40, the system prompts the second technician to verify the patch removal and wearer identity via a fourth electronic signature 46. The fourth electronic signature 46 may serve as an acknowledgment that the patch 14 has been removed and no tampering was evident. The electronic signature process for the second technician is initiated by generating a unique one-time-use code, which can be accessed by the second technician through various methods, including but not limited to, a scannable QR code displayed on the second technician's device, a text message link sent to the second technician's phone, or an email link sent to the second technician. Once the second technician accesses the electronic signature via a common web browser, the second technician is then prompted to use the system-generated signature.

Upon collecting the fourth electronic signature from the second technician, the system applies the fourth electronic signature identifier 48 to the chain of custody document 36 (FIG. 10B) and displays the same to the second technician for signature and confirmation to signature categories, wherein the second technician may review the content and apply their electronic signature 46 using a highlighted visual cue to ensure the correct signature placement and thereby authenticate and authorize the electronic signature 46, at which point the second technician's credentials are verified and entered into the chain of custody document 36. After reviewing and confirming the details, and authorization of the fourth electronic signature 46, the second technician clicks a complete button to finalize their portion of the electronic signature process and generate a fourth electronic signature identifier to be securely stored in the user interface database and linked with the third electronic signature identifier 48, the unique client identifier 12, the unique case record identifier 15, and the patch serial number 16.

Once the removal event is logged and the third and fourth electronic signatures 40, 46 are secured, at step 111, a completed chain of custody document 36 is compiled, wherein the chain of custody document comprises a unique barcode 52 associated with each of unique client identifier 12, the unique case record identifier 15, the patch serial number 16, the first electronic signature identifier 22, the second electronic signature identifier 32, the third electronic signature identifier 42 and the fourth electronic signature identifier 48. Once the completed chain of custody document 36 is compiled the same is uploaded and recorded on the user interface database in connection with the pertinent case record and client profile, as shown in FIG. 10C, such that details and information associated therewith are stored and available for access at any time.

At step 112, upon compilation of the completed chain of custody document 36, the same may be printed from the system. The completed chain of custody document 36 may contain all relevant patch application and removal information. The completed chain of custody document 36 may also include identification seals 54 (FIG. 10B) printed with unique barcode identifiers 52 or other security identifiers that correspond to the respective unique client identifier 12, the unique case record identifier 15, the patch serial number 16, the first electronic signature identifier 22, the second electronic signature identifier 32, the third electronic signature identifier 42 and the fourth electronic signature identifier 46.

As detailed herein above, in step 108A, once the completed chain of custody document 36 is printed, the specimen (i.e., the worn drug patch 14) may be securely sealed within a closed receptacle. At step 113, the closed receptacle may be sealed with one or more of the identification seals 54 for shipment to a designated laboratory for testing. To ensure the integrity of the specimen, it is contemplated that both the wearer and the second technician may be required to provide wet ink signatures on the security seals 54, ensuring acknowledgment by the wearer and the second technician of the proper handling and sealing of the drug patch 14.

Once the system compiles the completed chain of custody document 36 the system may then optionally notify the case manager, such as a parole officer or other agent, via a push notification generated from the user interface database and transmitted to the agent's mobile electronic device, that the drug patch 14 has been removed and sent to the laboratory for evaluation and analysis.

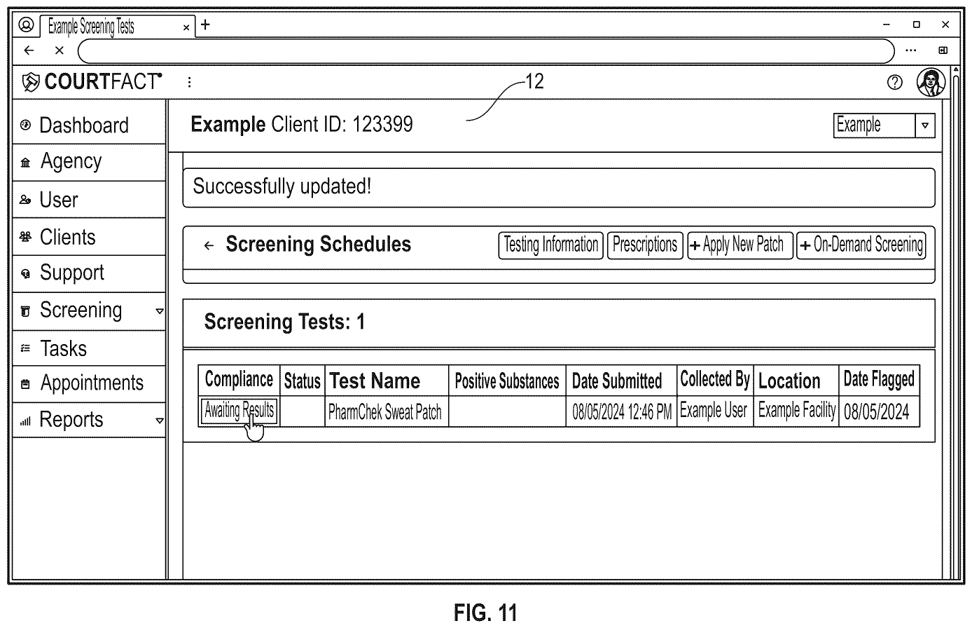

Once the specimen is sealed and prepared for shipment, the system is equipped to transmit all relevant information in the case record 15 electronically to the designated laboratory (FIG. 11), eliminating the need for manual data entry. When the sample arrives at the laboratory, the laboratory technician may scan the barcode identifier 52 printed on the identification seal 54 to gain access to the case record. This barcode identifier 52 is linked to the unique identifiers 12, 15, 16, 22, 32, 42, 46 generated during the patch application and removal events. Upon scanning, the laboratory information management system (LIMS) may automatically populate with all necessary specimen data, including the selected testing protocol 38. This integration minimizes the risk of errors in testing process.

At step 114, the laboratory transmits to the system a result of the testing protocol and the system links the result of the testing protocol with the unique client identifier 12, the unique case record identifier 15, the patch serial number 16, and the completed chain of custody document 36 in the user interface database and stores the result in the user interface database.

Once the result of the testing protocol is linked with the unique client identifier 12, the unique case record identifier 15, the patch serial number 16, and the completed chain of custody document 36 in the user interface database and stored in the user interface database, the system may optionally, at step 115, notify the case manager and the wearer, and other authorized third-parties, that the result of the testing protocol is available within the case record. More particularly, the system may notify the case manager, via a push notification generated from the user interface database and transmitted to the agent mobile electronic device and notify the wearer, via a push notification generated from the user interface database and transmitted to the wearer mobile electronic device, that the result of the testing protocol is recorded on the user interface database and available for review within the respective case record.

In the manner described above, the system and method as detailed in steps 101-115 of FIG. 1 are employed to electronically track the application and removal of a drug testing patch 14 on a wearer. It is also contemplated that the system provides for split electronic and wet signature functionality, allowing flexibility in situations where full electronic processing may not be feasible.

In one nonlimiting example, if a second technician must remove a drug testing patch 14 from a wearer where access to computers, the internet, or printing may be restricted, the technician is equipped to print the first portion of the chain of custody document 36 with the captured first electronic signature 20 and second electronic signature 30, with the remaining steps of the patch removal process, including the securing the third electronic signature 40 from the wearer and the fourth electronic signature 46 from the second technician as wet ink signatures. Once the patch has been removed and the wet signatures obtained, the second technician may manually update the system at a later time. Accordingly, the system may provide a manual input option to fill in associated details and information that were not collected electronically, such as the patch condition or removal details. The user can also upload into the case record a completed version of the chain of custody form 36, including any additional signatures or physical documentation generated during the patch removal event.

Moreover, it is contemplated that the system is equipped with a fallback provision for cases where electronic processes are unavailable or impractical. In such scenarios, the user can revert to a fully manual process, entering information from handwritten forms. This includes uploading photographic evidence, handwritten chain of custody documentation, and any other relevant data manually into the case record. The system allows all collected information to be digitized and tracked electronically once entered.

With regard to the media, processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claimed invention.

The detailed description and the drawings or figures are supportive and descriptive of the present teachings, but the scope of the present teachings is defined solely by the claims. While some of the best modes and other embodiments for carrying out the present teachings have been described in detail, various alternative designs and embodiments exist for practicing the present teachings defined in the appended claims.

While various embodiments have been described, the description is intended to be exemplary, rather than limiting, and it will be apparent to those of ordinary skill in the art that many more embodiments and implementations are possible that are within the scope of the embodiments. Any feature of any embodiment may be used in combination with or substituted for any other feature or element in any other embodiment unless specifically restricted. Accordingly, the embodiments are not to be restricted except in light of the attached claims and their equivalents. Also, various modifications and changes may be made within the scope of the attached claims.

Benefits, other advantages, and solutions to problems, and any element or elements that may cause any benefit, advantage, or solution to occur or become more pronounced, however, are not to be construed as critical, required, or essential features or elements of any or all of the claims, unless such benefits, advantages, solutions, or elements are expressly stated in such claims.

What is claimed is:

1. A method for tracking and verification of a periodized drug testing using a wearable drug testing patch, the method comprising:
creating a unique client profile and storing the unique client profile on a user interface database written on the non-transitory computer readable medium, wherein the unique client profile is assigned a unique client identifier;
opening a case record associated with the application of the wearable drug testing patch having a patch serial number, and storing the case record in the user interface database, wherein the case record is assigned a unique case record identifier;
applying, at a first facility, the wearable drug testing patch to a wearer by a first technician and logging a patch application event into the user interface database, wherein logging a patch application event into the user interface database further comprises:
requesting the verification of patch application and wearer identity via a first electronic signature from the wearer and assigning a first electronic signature identifier;
requesting the verification of patch application and wearer identity via a second electronic signature from the first technician and assigning a second electronic signature identifier;
linking the unique client identifier, the unique case record identifier, the patch serial number, the first electronic signature identifier, and the second electronic signature identifier in the user interface database; and
compiling an application chain of custody document for the linked unique client profile, the unique case record identifier, the patch serial number, the first electronic signature identifier, and the second electronic signature identifier and storing the same in the user interface database.

2. The method of claim 1 wherein the wearable drug testing patch is worn by the wearer for a predetermined duration following the application of the wearable drug testing patch to the wearer at the first location by the first technician; and wherein the predetermined duration is from about three (3) days to about fourteen (14) days.

3. The method of claim 2 wherein the predetermined duration is about seven (7) days.

4. The method of claim 1, wherein the step of applying, at a first facility, the wearable drug testing patch to the wearer by the first technician further comprises:
capturing, via a capture device, at least one image of the wearable drug testing patch as applied to the wearer at the time of application; and
storing the at least one image in the user interface database, such that the at least one image is configured to confirm the location of placement of the wearable drug testing patch upon the wearer at the time of application and the patch serial number.

5. The method of claim 4 wherein the at least one image includes a face of the wearer such that the identity of the wearer may be visually verified.

6. The method of claim 4 wherein the at least one image is date stamped and time stamped.

7. The method of claim 6 further comprising the step of:
notifying a case manager, via a push notification generated from the user interface database and transmitted to an agent mobile electronic device, that the wearable drug testing patch has been applied to the wearer and the predetermined duration for which the wearable drug testing patch will be affixed to the wearer.

8. The method of claim 6 further comprising:
notifying the wearer, via a push notification generated from the user interface database and transmitted to a wearer mobile electronic device, that the predetermined duration has lapsed and that the wearable drug testing patch is due for removal.

9. The method of claim 8 further comprising:
determining a current location of the wearer via a set of Global Positioning System (GPS) coordinates associated with the wearer mobile electronic device;
populating a list of removal locations that are geographically proximate to the wearer; and
identifying and selecting a second facility for removal of the wearable drug testing patch from the list of removal locations that are geographically proximate to the wearer.

10. The method of claim 8 further comprising the steps of:
accessing the case record associated with the application of the wearable drug testing patch in the user interface database via the unique case record identifier; and
removing, at a second facility, the wearable drug testing patch from the wearer by a second technician, after the predetermined duration has elapsed.

11. The method of claim 10, wherein the step of removing, at a second facility, the wearable drug testing patch from the wearer by a second technician, after the predetermined duration has elapsed further comprises:
capturing, via a capture device, at least one image of the wearable drug testing patch as applied to the wearer at the time of removal; and
storing the at least one image in the user interface database, such that the at least one image is configured to confirm the location of placement of the wearable drug testing patch upon the wearer at the time of removal and the patch serial number.

12. The method of claim 11, wherein the at least one image is date stamped and time stamped.

13. The method of claim 11, wherein the at least one image includes a face of the wearer such that the identity of the wearer may be visually verified.

14. The method of claim 12 further comprising the step of securing the wearable drug testing patch in a closed receptacle.

15. The method of claim 14 further comprising:
selecting the testing protocol for the wearable drug testing patch; and
linking the selected testing protocol to the unique case record identifier in the user interface database.

16. The method of claim 15 wherein:
the wearable drug testing patch is configured to detect at least one drug metabolite selected from the group consisting of amphetamine, methamphetamine, cocaine, opiates, marijuana, fentanyl, norfentanyl, phencyclidine, hydrocodone, oxymorphone, oxycodone, benzodiazepines, methadone, buprenorphine, and norbuprenorphine; and
the testing protocol is configured to include groupings of tests associated with at least one of amphetamine, methamphetamine, cocaine, opiates, marijuana, fentanyl, norfentanyl, phencyclidine, hydrocodone, oxymorphone, oxycodone, benzodiazepines, methadone, buprenorphine, and norbuprenorphine.

17. The method of claim 16 further comprising the steps of:
requesting the verification of patch removal and wearer identity via a third electronic signature from the wearer and assigning a third electronic signature identifier;
requesting the verification of patch removal and wearer identity via a fourth electronic signature from a second technician and assigning a fourth electronic signature identifier;
linking the third electronic signature identifier and the fourth electronic signature identifier to the case record in the user interface database; and
compiling a completed chain of custody document for the case record, wherein the chain of custody document comprises a unique barcode associated with each of unique client identifier, the unique case record identifier, the wearable drug testing patch serial number, the first electronic signature identifier, the second electronic signature identifier, the third electronic signature identifier, and the fourth electronic signature identifier.

18. The method of claim 17 wherein the first facility and the second facility are the same.

19. The method of claim 17 wherein the first facility and the second facility are different.

20. The method of claim 17 wherein the first technician and the second technician are the same.

21. The method of claim 17 wherein the first technician and the second technician are different.

22. The method of claim 17 further comprising the step of:
printing the completed chain of custody document;
removing a barcode sticker comprising the unique barcode thereon from the completed chain of custody document; and
sealing the closed receptacle with the bar code sticker in the presence of the wearer and the second technician.

23. The method of claim 22 further comprising the steps of:
capturing a first wet signature from the second technician to verify the sealing of the wearable drug testing patch in the closed receptacle; and
capturing a second wet signature from the wearer to verify the sealing of the wearable drug testing patch in the closed receptacle.

24. The method of claim 23 further comprising the steps of:
notifying the case manager, via a push notification generated from the user interface database and transmitted to the agent mobile electronic device, that the drug patch has been removed.

25. The method of claim 23 further comprising the steps of:
transmitting the closed receptacle containing the removed wearable drug testing patch to a selected laboratory for analysis consistent with the selected testing protocol; and
providing access by the laboratory to the case record, via scanning of the unique barcode.

26. The method of claim 25 further comprising the steps of:
receiving a result of the testing protocol from the laboratory; and
linking the result of the testing protocol with the unique client identifier, the unique case record identifier, the patch serial number, and the chain of custody document in the user interface database and storing the results in the user interface database.

27. The method of claim 26 further comprising the steps of:
providing access to the user interface database by the laboratory, the wearer, and the case manager to display and transmit the result of the testing protocol.

28. The method of claim 27 further comprising the steps of:
notifying the case manager, via a push notification generated from the user interface database and transmitted to the agent mobile electronic device, that the result of the testing protocol is available on the user interface database; and
notifying the wearer, via a push notification generated from the user interface database and transmitted to the wearer mobile electronic device, that the result of the testing protocol is available on the user interface database.
